(19) United States
(12) Patent Application Publication
(10) Pub. No.: US 2007/0225820 A1
(43) Pub. Date: Sep. 27, 2007

(54) TOTAL WRIST PROSTHESIS

(75) Inventors: Gareth Thomas, Dorset (GB); John Knowles Stanley, Lancashire (GB)

Correspondence Address: FITCH EVEN TABIN AND FLANNERY, 120 SOUTH LA SALLE STREET, SUITE 1600, CHICAGO, IL, 60603-3406, US

(73) Assignee: ASCENSION ORTHOPEDICS, INC., Austin, TX (US)

(21) Appl. No.: 11/750,695

(22) Filed: May 18, 2007

Related U.S. Application Data
(62) Division of application No. 10/746,982, filed on Dec. 23, 2003, now abandoned.

(51) Int. Cl. A61F 2/42

(52) U.S. Cl. 623/21.12

(57) ABSTRACT

Total wrist prosthesis comprising a radial component with a stem for fixation into the radius, a carpal component with one or more stems for fixation into the capitate and an intermediate bearing component which articulates with the radial component and allows rotational movement of the carpal component relative to the radial component plus hinged movement in two transverse planes. The radial component body may have a concave or convex surface to articulate with a matching surface on the intermediate bearing component. The carpal body may have a projection which is received in a recess in a base surface of the intermediate bearing component, and which has a pair of lugs that are received in an annular groove in the recess for an axially secure connection that allows small relative rotational movement as permitted by tendons and ligaments of the wrist.

This application is a divisional of U.S. application Ser. No. 10/746,982 filed Dec. 23, 2003, the disclosure of which is incorporated herein by reference.

### TECHNICAL FIELD OF THE INVENTION

The present invention relates to a total wrist prosthesis.

### BACKGROUND OF THE INVENTION

Various wrist prostheses have hitherto been known. Many of these allow for movement in two planes, namely flexion and extension movement in a first plane, and ulnar deviation and radial deviation in another plane. However, provision is missing for additional small rotational movements of the hand relative to the arm in a third plane. Also some prosthesis parts may be joined too loosely and may lead to disjointing problems.

### SUMMARY OF THE INVENTION

The invention seeks to provide a prosthesis which allows hand movements in three planes and which provides a reliable joint, reducing the chances of dislocation and/or loosening.

According to a particular embodiment of the present invention there is provided a total wrist prosthesis comprising;

a) a proximal radial component having a radial body and a stem extending from one side of the body that allows fixation into the resected distal end of the radius,

b) a distal carpal component having a carpal body and one or more stems extending from one side of the body that allows fixation into the capitate, and

c) an intermediate bearing component that is connected to the carpal component so as to allow rotational movement of the carpal component relative to the intermediate bearing component and thereby allow small rotational movement of the hand relative to the arm, and that articulates with a facing surface of the radial component so as to allow only hinged movement between the intermediate bearing component and the radial component in two transverse planes so as to allow for flexion and extension movement of the hand in a first plane and ulnar and radial deviation in a second transverse plane.

In one embodiment, the carpal component has one or more apertures to receive screws for fixation into one or more carpals.

In one embodiment, the radial body has a double-curved concave or convex surface on its side remote from its shaft to engage with a matching convex or concave surface on the intermediate bearing component.

In one embodiment the radial body has a toroidal or ellipsoidal concave recess which engages and articulates with a toroidal or ellipsoidal convex surface on the intermediate bearing component. The carpal body may have a projection on its side remote from its stem which is rotatively received in a recess in the intermediate bearing component. In one embodiment, the projection is generally cylindrical, having a pair of opposed lugs extending from the cylindrical wall thereof, the projections are received and axially constrained in an annular groove in the recess of the intermediate bearing component to prevent separation of the carpal component from the intermediate bearing component. The projecting lugs may slide through diametrically opposed keyways in the intermediate bearing component to reach the groove; rotation then assembles the intermediate bearing component with carpal component.

### BRIEF DESCRIPTION OF THE DRAWINGS

An embodiment of the invention will now be described with reference to the accompanying drawings in which:

FIGS. 1A and 1B show a perspective exploded view of a prosthesis from either end,

FIG. 2 shows a front view of FIG. 1,

FIG. 3 shows a side view of FIG. 1,

FIG. 4 shows a collar and screw in an aperture of part of a prosthesis,

FIG. 5 shows a cross section, front and end view of the collar, and

FIG. 6 shows a side and end view of the screw.

### DETAILED DESCRIPTION OF THE PREFERRED EMBODIMENTS

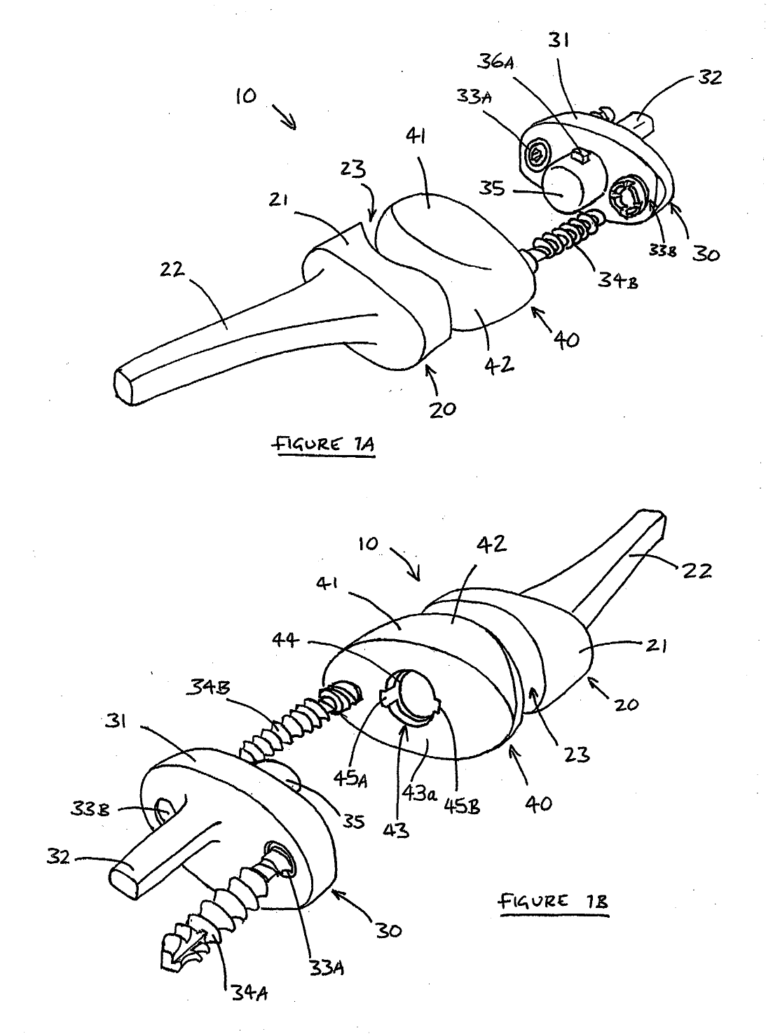

FIGS. 1 to 3 show a prosthesis 10 which comprises a proximal radial component 20, a distal carpal component 30, and an intermediate bearing component 40.

Proximal radial component 20 may be formed from any suitable material such as a cobalt chrome alloy. Radial component 20 has a generally oval body 21 and a stem 22 extending from one side of the body that allows fixation into the resected distal end of the radius. Part of the body and/or shaft may be coated with a textured bone ingrowth surface to provide for more secure joinder to the bone.

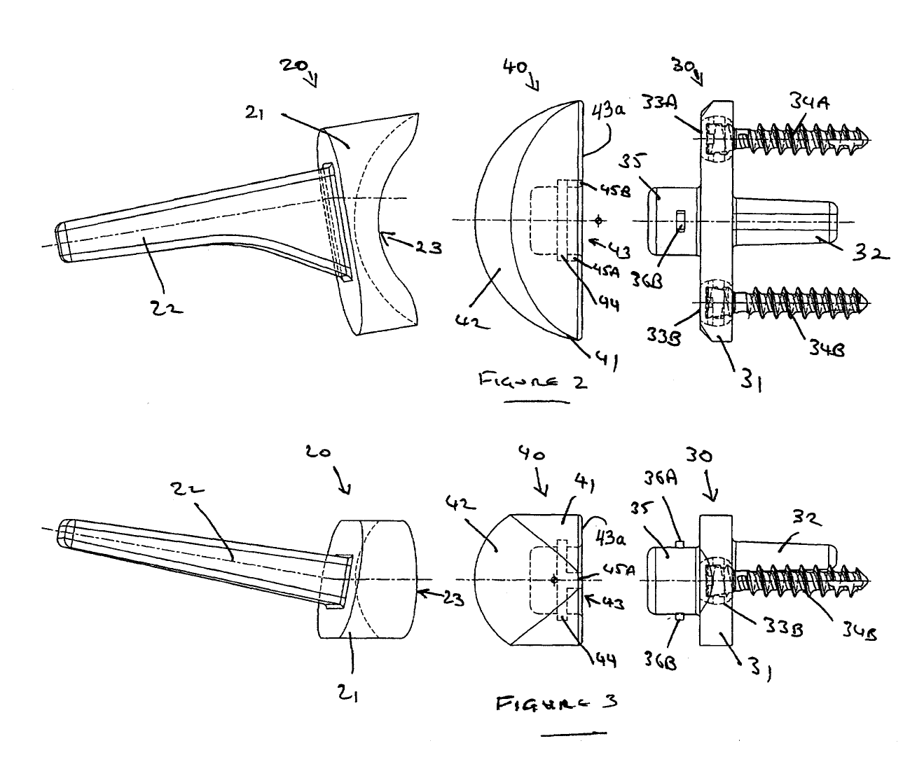

Radial body 21 has a toroidal or ellipsoidal concave surface 23 on its side remote from its stem 22. This double-curved surface 23 has its larger radius of curvature in the direction of body length and its smaller radius of curvature in the direction of body width, normal to the larger radius of curvature. Also as clear from the dotted lines in FIGS. 2 and 3, one side and one end wall of the body 21 may be higher than its opposite side and end wall; such may provide a 10 degree slope across the two side walls and two end walls to reduce potential subluxion of the implanted prosthesis.

Distal carpal component 30 may also be formed from any suitable material such as a cobalt chrome alloy. Carpal component 30 has a generally oval carpal body 31 and a stem 32 extending from one surface of the body 31 which facilitates location into the capitate. A pair of apertures 33A, 33B are provided in the body either side of stem 32 to receive screws that provide stabilization and fixation into the flanking carpal bones. Such screws may be locking screws, e.g. of the type described later in more detail. Accordingly apertures 33A, 33B may be adapted to receive screws with collars 34A 34B of the type described and shown herein or other suitable screws. Again, part of the body and/or the stem 32 may be coated with a textured bone ingrowth surface to provide bone ingrowth.

The carpal body 31 has a projection 35 on its opposite surface remote from its stem 32. The projection 35 has a sidewall that is a surface of revolution; it may be cylindrical or frustoconical in shape. A pair of opposed lugs 36A, 36B extend from the projection sidewall.

Intermediate bearing component 40 has a body 41 of oval perimeter formed from ultra high molecular weight polyethylene to provide a low motion bearing surface interfacing with cobalt chrome. The body 41 has a main convex double-curved surface 42 which is proportioned to articulate with the facing concave surface 23 of the radial component. As shown in FIGS. 2 and 3, the convex toroidal or ellipsoidal surface 42 also has its larger radius of curvature over the body length and its smaller radius of curvature over the body width, normal to the larger radius of curvature; these correspond to substantially the same radii of curvature of the concave surface 23 of the radial body 21.

In use, the double-curved convex surface 42 and mating concave surface 23 allow only hinged relative movement between the intermediate bearing component and the radial component in two transverse planes so as to allow for flexion and extension movement of the hand in a first plane and ulnar and radial deviation in a second perpendicular plane.

On the side of body 41 opposite to the convex surface 42, there is a base surface 43a wherein there is a recess 43 which includes an intermediate annular groove 44 (see FIG. 2). The recess 43 has an interior wall which is a surface of revolution that matches the shape of the projection, e.g. cylindrical or frustoconical. Keyways 45A, 45B provide diametrically opposed passageways leading from the base surface 43a to the intermediate annular groove 44. The intermediate bearing component 40 is releasably connected to the carpal body 31 by entry of the projection 35 into mating recess 43, with lugs 36A, 36B sliding in keyways 45A, 45B, followed by rotation of the body 41 through 90 degrees so that lugs 36A, 36B enter and reside in the annular groove 44 where they are axially restrained so that separation of the intermediate bearing component from the carpal component is prevented. The lugs do not rotationally restrict relative movement of the bearing component 40, and in use, this is important as it allows rotational movement of the intermediate bearing component relative to the carpal component whereby to allow small rotational movements of the hand relative to the arm in a third transverse plane, to an extent permitted by the tendons and ligaments of the wrist.

The invention may take a form other than that specifically described above. For example the double-curved concave surface could be formed on the intermediate bearing component and the matching convex surface formed on the radial component. Also other types of joints could be used to allow the slight rotational movement of the intermediate bearing component relative to the carpal component.

Angling of the shaft 22 provides good anatomical compliance and supports the prosthesis during flexion and extension to prevent volar dislocation. Also the toroidal or ellipsoidal matching surfaces on the intermediate bearing component and the radial component is designed to mimic anatomic movement.

Locking bone screws are preferably used to secure prostheses to bones, such as locking screws having two components. One component is an internally-threaded annular collar with a circumferential cut out allowing the collar to expand and contract. The collar has an arcuate outer surface to engage and rotate in an arcuate wall of an aperture in a prosthesis. The other component is a screw having a continuous and constant thread extending from a screw shank which tapers outwards towards the screw head. In use the collar is inserted in the prosthesis and the screw placed into threaded engagement with the collar. The screw can then be screwed into the bone. Before the screw is screwed fully into the bone and collar, the screw can toggle relative to the prosthesis so its angle can change as the collar can rotate in the aperture of the prosthesis. However when the screw is fully screwed into the bone and collar, the widened taper of the shank of the screw expands the collars so locking the collar in the aperture and the screw in the collar.

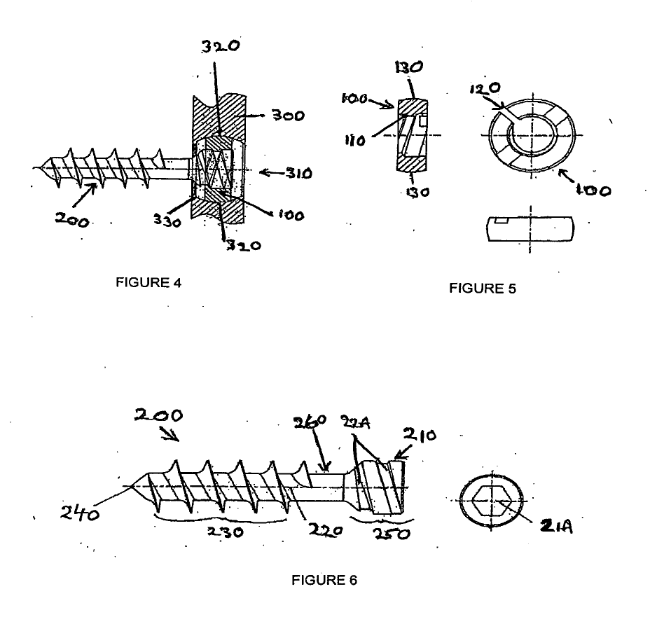

A problem with this known arrangement is that an ideal screw thread for engaging bone tissue is not always the ideal screw thread for engaging the collar. Shown in FIGS. 4-6 is a locking screw comprising:

a) an internally-threaded annular collar with a circumferential cut out allowing the collar to expand and contract, said collar having an arcuate outer surface to engage and rotate in an arcuate wall of an aperture;

b) a screw having a head at one end of a screw shank, a first thread on the screw shank extending from the screw end remote from the head towards the head, a second thread on the shank adjacent the head, and a threadless section of shank or lag between the first and second thread, said shank supporting the second thread being tapered such that it expands the collar when screwed into the collar.

The locking screw may be a locking bone screw.

The first thread may be a cancellous thread such as an ISO standard bone thread.

The second thread may be a machine thread such as an ISO machine thread.

The invention also extends to a prosthesis with an aperture with an arcuate wall incorporating the above defined locking bone screw. The aperture may have an annular flange at one end of the aperture to prevent the collar passing through said end of the aperture.

An embodiment of the locking screw is described with reference to FIGS. 4-6.

Referring to FIG. 4 there is provided a locking bone screw having an annular collar 100 and screw 200 in an aperture 310 of a prosthesis 300.

Referring more particularly to FIG. 5, annular collar 100 has an internal thread 110. A circumferential cut out 120 is provided allowing the collar to expand and contract. Collar 100 may be formed of resilient material such as metal, e.g. titanium-based metal. Collar 100 has an arcuate outer surface 130 to engage and rotate in an arcuate wall 320 of aperture 310 in prosthesis 300.

As shown in FIG. 6, screw 200 has a head 210 at one end of a screw shank 220. Screw 200 may be of metal such as titanium-based metal. Screw head may have a hexagonal recess 21A to receive a tool (not shown) to rotate the screw.

A first thread 230 on the screw shank extends from the screw end 240 remote from the head 210 towards the head 210. First thread 230 is a cancellous thread such as an ISO standard bone thread.

A second thread 250 is provided on the shank 220 adjacent the head. The shank section 22A supporting thread 250 tapers outwards toward the end of the head 210. The second thread 250 is a machine thread such as an ISO machine thread.

A threadless section or lag 260 of the shank is provided between the first and second thread.

First thread 230 and second thread 250 are of a size to engage with the internal thread 110 of collar 100. However when first thread 230 is engaged with internals thread 110 of collar 100, collar 100 is able to rotate in aperture 310 allowing the screw 200 to toggle relative to the prosthesis. When second thread 250 is fully engaged with internal thread 110 (e.g. the screw 200 is fully screwed into collar 100 as shown in FIG. 4), the tapered shaft supporting thread 250 expands collar 100 so its arcuate outer surface 130 locks against the arcuate wall 320 of aperture 310 in prosthesis 300.

It is preferred that an annular flange 330 is provided at one end of the aperture 310 to prevent the collar passing through this end of the aperture, so preventing separation of the screw 200 from the prosthesis 300. Flange 330 also limits rotation of the collar 100 in aperture 310.

In use, screw 200 is placed in collar 100 in the prosthesis aperture 310 and is screwed into a bone using a tool with the thread 230 engaging the bone tissue. The desired angle of the screw is achieved as it can toggle relative to the prosthesis. The collar 100 finally locks in the aperture 310 as the head 210 is screwed into the collar.

Because the thread 250 is a machine thread, it engages well with the metal collar to prevent it unscrewing itself, and the bone thread 230 bonds well with bone.

Once the prosthesis is connected to the bone for a period of time, fibrous growth forms around the threadless section or lag in shank 220 which helps prevent the screw from unscrewing out of the bone.

The above-described locking screw may be used separately or in conjunction with the prosthesis 10. The locking bone screw may have applications outside medical application, i.e. used to secure fittings to material other than bones.

Further modifications will be apparent to those skilled in the art without departing from the scope of the present invention.

1. A total wrist prosthesis comprising:
a) a proximal radial component having a radial body and a stem extending from one side of the body that allows fixation into the resected distal end of the radius,
b) a distal carpal component having a carpal body and one or more stems extending from one surface of the body that allows fixation into the capitate, and
c) an intermediate bearing component which is connected to the carpal component so as to allow rotational movement of the carpal component relative to the intermediate bearing component thereby permitting small rotational movements of the hand relative to the arm, the intermediate bearing component articulating with a surface of the radial component opposite said one side so as to allow only hinged movement in two transverse planes between the intermediate bearing component and the radial component so as to allow for flexion and extension movement of the hand in a first plane and ulnar and radial deviation of the hand in a second transverse plane.

2. A total wrist prosthesis according to claim 1, wherein the carpal component has one or more apertures to receive screws for fixation into one or more carpals.

3. A total wrist prosthesis according to claim 1, wherein the surface of the radial body on its side remote from its stem is a toroidal or ellipsoidal concave or convex surface which articulates with a matching surface on the intermediate bearing component.

4. A total wrist prosthesis according to claim 3, wherein the radial body surface is a concave surface which articulates with a matching convex surface on the intermediate bearing component.

5 . A total wrist prosthesis according to claim 4, wherein the carpal body has a projection extending from its surface opposite from said one surface, which projection is relatively rotatably received in a recess in a base surface of the intermediate bearing component.

6. A total wrist prosthesis according to claim 5, wherein the projection is generally cylindrical and has a pair of diametrically opposed lugs extending from the sidewall thereof, said projections being received and axially engaged in an annular groove in the recess of the intermediate bearing component so as to prevent axial separation of the intermediate bearing component from the carpal component while permitting small relative rotational movement about the projection in the implanted prosthesis.

7. A total wrist prosthesis according to claim 6, wherein the lugs have access to the annular groove through a pair of keyways in the intermediate bearing component that flank the recess and extend from the base surface to the groove.

8. A total wrist prosthesis comprising:
a) a proximal radial component having a radial body and a stem extending from one side of the body that allows fixation into the resected distal end of the radius, which body has a concave surface on its opposite side,
b) a distal carpal component having a carpal body and one or more stems extending from one surface of the body that allows fixation into the capitate, and
c) an intermediate bearing component which is connected to the carpal component at an opposite surface to said one surface so as to allow relative rotational movement between the carpal component and the intermediate bearing component thereby permitting small rotational movements of the hand relative to the arm, the intermediate bearing component having a convex surface which articulates with the concave surface of the radial component, which articular concave and convex surfaces are shaped so as to allow only hinged movement between the intermediate bearing component and the radial component in two transverse planes, permitting flexion and extension movement of the hand in a first plane and ulnar and radial deviation of the hand in a second transverse plane.

9. A total wrist prosthesis according to claim 8, wherein the radial body concave surface is a toroidal or ellipsoidal surface and the intermediate bearing component convex surface is a matching toroidal or ellipsoidal surface.

10. A total wrist prosthesis according to claim 9, wherein the carpal body has a projection on its surface opposite from said one surface, which projection is received in a recess in a base surface of the intermediate bearing component and defines relative rotation of the intermediate bearing component and the carpal body.

11. A total wrist prosthesis according to claim 10, wherein the projection has a sidewall which is a surface of revolution and has a pair of diametrically opposed lugs extending from the sidewall, said projections being received and axially engaged in an annular groove in the recess of the intermediate bearing component so as to prevent axial separation of the intermediate bearing component from the carpal component while permitting small relative rotational movement about the projection in the implanted prosthesis.

12. A total wrist prosthesis according to claim 11, wherein the lugs have access to the annular groove through a pair of keyways in the intermediate bearing component that flank the recess and extend from the base surface to the groove.

13. A total wrist prosthesis according to claim 11 wherein said projection has a cylindrical sidewall.

14. A total wrist prosthesis according to claim 10 wherein the base surface and the opposite surface of the carpal body are juxtaposed and of similar shape so as to permit slidable rotational relative movement therebetween.

15. A total wrist prosthesis according to claim 14, wherein the carpal body has one or more apertures to receive screws for fixation into one or more carpals with heads of such screws recessed within the body below the opposite surface thereof.
